(19) United States
(12) Patent Application Publication
(10) Pub. No.: US 2002/0150912 A1
(43) Pub. Date: Oct. 17, 2002

(54) Reporter system for cell surface receptor-ligand binding

(76) Inventors: Christer S. O. Owman, Lund (SE); Bjorn A. Olde, Lund (SE); Knut Kotarsky, Lund (SE)

Correspondence Address: Finnegan, Henderson, Farabow,, Garrett & Dunner, L.L.P., 1300 I Street, N.W., Washington, DC, 20005-3315, US

(21) Appl. No.: 09/946,334

(22) Filed: Sep. 6, 2001

Related U.S. Application Data
(60) Provisional application No. 60/230,705, filed on Sep. 7, 2000.

(51) Int. Cl. C12Q 1/68; G01N 33/53; G01N 33/567; C12P 21/02; C12N 5/06

(52) U.S. Cl. 435/6; 435/325; 435/320.1; 435/7.2

(57) ABSTRACT

The present invention provides chimeric reporter constructs, recombinant cells containing the reporter constructs, and assays utilizing the recombinant cells for detection of substances that interact with cell surface receptors, such as those of the G-protein coupled receptor family. The reporter constructs and recombinant cells are particularly well suited for high-throughput screening assays, and detection of interaction between a substance and a cell surface receptor can be performed visually, by FACS, or by luminometry.

### CROSS-REFERENCE TO RELATED APPLICATION

This application relies on, and claims the benefit of U.S. Provisional application Serial No. 60/230,705, filed Sep. 7, 2000, the entire disclosure of which is incorporated herein by reference.

### BACKGROUND OF THE INVENTION

1. Field of the Invention

This invention relates to the field of recombinant nucleic acid technology. It further relates to the field of drug discovery. More particularly, this invention relates to recombinant nucleic acids, recombinant cells, kits, and assays for detection of substances that interact with cell surface receptors, such as G-protein coupled receptors (GPCRs), tyrosine kinase-type receptors, and ion channels. The recombinant nucleic acids, recombinant cells, kits, and assays are well suited for high-throughput screening (HTS).

2. Description of the State of the Art

Various assays for detecting substances that interact with cell surface receptors are known in the art. Generally, these assays rely on recombinant cells that express a receptor of interest, and link interaction of a substance and the receptor to up- or down- regulation of a reporter gene. The goal of many of these assays is to identify substances that are pharmaceutically active. Such pharmaceutically active substances can be used as drugs to counteract undesirable over- or under-expression of a given signal pathway, which may be associated with a disease state or disorder.

For example, U.S. Pat. No. 5,401,629 to Harpold et al. discloses recombinant cells and assay systems for assaying compounds for their agonist or antagonist activity on ion channels and/or cell surface receptors. The '629 patent discloses a recombinant cell having receptors on its cell surface that is transformed with a reporter gene construct. The construct comprises 1) a transcriptional control element that is responsive to an intracellular condition that occurs when the receptor interacts with a compound having agonist or antagonist activity for the receptor, and 2) a reporter gene encoding a detectable gene product, where the reporter gene is operatively associated with the transcriptional control element. The transcriptional control element is responsive to calcium, cAMP, or NGF. The receptor to be assayed is a G-protein coupled receptor, such as adrenergic receptors, and muscarinic receptors. Reporters are CAT, firefly luciferase, bacterial luciferase, and alkaline phosphatase. The cell line must be capable of transfection, and have low or no background levels of the specific receptor of interest. Receptors are listed at column 5, line 42 through column 6, line 12. The examples disclose recombinant mammalian cells and assays. However, the assays of the '629 patent rely on time-consuming and labor-intensive clonal selection methods to identify and obtain cells having high levels of expression. In addition, the assays suffer from high levels of background signal, which reduces the sensitivity of the assay.

U.S. Pat. No. 5,436,128 to Harpold et al. discloses methods for detecting and identifying substances that act as agonists or antagonists of specific cell surface localized receptors and ion channels, as well as recombinant cells useful in the methods. The recombinant cells of the '128 patent are genetically engineered to express specific ion channels or cell surface receptors, and also contain DNA constructs that include a reporter gene coupled to a regulatory region that is controlled by signals originating from the receptor or ion channel. The recombinant cells can endogenously express the cell surface protein or can express heterologous DNA that encodes the cell surface protein. The cell surface receptor is a G-protein coupled receptor, such as a muscarinic receptor. The regulatory region comprises regulatory sequences from the c-fos gene, the VIP gene, the somatostatin gene, the proenkephalin gene, the carboxykinase gene, and the nerve growth factor-1 gene, as well as cAMP responsive elements and elements responsive to intracellular calcium ion levels. The reporter gene is CAT, firefly luciferase, bacterial luciferase, β-galactosidase, or alkaline phosphatase. The examples disclose recombinant mammalian cell lines and assays. However, as with the assays of the '629 patent, the assays of the '128 patent require clonal selection methods that are time consuming, and the assays suffer from high levels of background signal.

U.S. Pat. No. 5,854,004 to Czernilofsky et al. discloses a process for screening substances having modulating effects on a receptor-dependent signal transmission pathway, and recombinant cells useful in such a process. The assay uses recombinant cells expressing G-protein coupled receptors. The recombinant cells contain a recombinant DNA encoding a reporter that is coupled to a regulatory sequence that responds to the change in an intracellular concentration of a molecule associated with activity of the receptor. The regulating molecule is inositol-1,4,5-triphosphate, diacylglycerol, cAMP, or calcium. The regulatory element is a TRE or CRE regulating element. Mammalian cells are disclosed as useful. Reporter genes are alkaline phosphatase, β-galactosidase, CAT, and luciferase. Receptors are the G-protein coupled receptors. However, as with the '629 and '128 patents, the clonal selection method of the '004 patent is time and labor intensive, and results in a high background-to-signal ratio.

Himmler et al. (Journal of Receptor Research, 13(1-4):79-94, 1993) discloses a cellular screening system that measures the biological activity of drugs acting on receptors. The system relies on coupling of the receptor to the cAMP signal transduction pathway to transcriptionally activate a reporter gene operative linked to multiple cAMP responsive elements (CREs). A stable recombinant cell line expressing the human dopamine D₁ receptor and luciferase under the control of CREs showed luciferase induction upon stimulation with apomorphine.

In addition, Weyer et al. (Receptors and Channels, 1:193-200, 1993) discloses a cellular assay system for the detection of substances that modulate the activity of G-protein coupled receptors by linking the expression of a reporter gene to activation of the G-protein coupled receptor through the phospholipase C system. Recombinant cells are disclosed that contain a luciferase gene under the control of the ICAM-1 gene regulatory region. These recombinant cells can further contain constructs that encode the human neurokinin 2 receptor or the human serotonin 2 receptor. Expression of the luciferase gene is controlled by interaction of molecules with the receptors encoded by the recombinant cells.

Several reporter systems have been described for receptors coupling to adenylate cyclase (Chen, W., et al., Anal. Biochem., 22:349-54, 1995) as well as for receptors that act by mobilizing Ca²⁺ (Weyer et al., supra; Stratowa, C., et al., J. Recept. Signal Transduct. Res., 15:617-30, 1995; Sista, P., et al., Mol. Cell. Biochem., 141:129-34, 1994; Schadlow, V., et al., Mol. Biol. Cell, 3:941-51, 1995). However, none of them has been optimized thoroughly for efficient mass screening of chemical compounds in varying milieus.

More recently, systems for detecting alterations in the activity of signal transduction pathways as a result of interaction of cell surface receptors and a substance have included dual reporter constructs. For example, Stables et al. (Journal of Receptor & Signal Transduction Research, 19(1-4):395-410, 1999) discloses the simultaneous use of two different luciferase reporters, each responsive to a different G-protein coupled receptor, for the detection of substances that interact with the receptors. In the assay, recombinant Chinese Hamster Ovary (CHO) cells expressing the human Vasopressin V₂ receptor and containing the firefly luciferase reporter gene operably coupled to a cAMP responsive element, were co-cultured with recombinant CHO cells expressing the human β₂-adrenoceptor and containing the Renilla luciferase reporter gene operably coupled to a cAMP responsive element. Because the firefly luciferase and Renilla luciferase activities depend on different substrates and reaction conditions, activation of one, for example as a result of the recombinant cells coming in contact with a substance that interacts with a recombinant receptor, can be differentiated from activation of the other. Thus, the assay can provide, from a single culture, information about whether a sample contains a substance that activates a single, or even multiple, specific G-protein coupled receptors. However, like the assays discussed above, the assay of Stables et al. utilizes time-consuming clonal selection methods to identify those cells that are most useful for the assay.

The superfamily of G-protein coupled receptors (GPCRs), or heptahelix receptors, is the most widely distributed among membrane receptors in eukaryotic cells (see, for example, Watson, S., and Arkinstall, S., The G-Protein Linked Receptor FactsBook, Academic Press, London, 1994). They receive signals from a large variety of substances from many different chemical classes, resulting in diverse intracellular, tissue, and organ responses. Among the various substances that interact with G-protein coupled receptors, the chemotactic substances form an extensive group. This group regulates the trafficking of immune cells during a microbial challenge. In addition, G-protein coupled chemokine receptors have recently received extensive interest because several of them are necessary for the HIV-1 virus to fuse with, and subsequently infect, CD4-positive cells (Weiss, R. A., and Clapham, P. R., Nature, 381:647-648, 1996; Hill, C. M., and Littman, D. R., Nature, 382:668-669, 1996; Fauci, A. S., Nature, 384:529-533, 1996). Other G-protein coupled receptors include muscarinic acetylcholine receptors, adrenergic receptors, serotonin receptors, and opsin receptors, as well as other neurotransmifter receptors and hormone receptors.

Members of the superfamily of G-protein coupled receptors constitute targets for more than 70% of the pharmaceutical drugs in current clinical use. Because of the multitude of physiological actions they mediate, a large proportion of drug testing is conducted on this kind of membrane receptor. The advent of high-throughput screening (HTS) has created a need for efficient cell-based reporter systems specially designed for GPCRs.

While recombinant G-protein coupled receptor assays are known, many of which are applicable to high-throughput screening, there still exists a need in the art for improved assays that are more sensitive and not as labor and time intensive.

### SUMMARY OF THE INVENTION

The present invention addresses shortcomings in the art by providing a rapid, reliable, relatively inexpensive reporter system that is amenable to high-throughput screening. The invention provides genetically engineered reporter systems that can be used to detect substances that interact with selected cell surface receptors. Thus, the invention provides new, optimized, cell-based reporter systems that are well suited for GPCRs that act through Ca²⁺ mobilization and signal through the mitogen-activated protein (MAPK) cascade.

The systems of the invention use recombinant cells containing reporter constructs in which a chimeric reporter gene is operably linked to at least one transcription control element, such as a second messenger-responsive element, such that activation or, by the inclusion of silencer motifs, repression of expression of the chimeric reporter gene occurs as an ultimate result of binding of a ligand to a cell surface receptor or interaction of a ligand with an ion channel on the surface of the recombinant cell. The reporter construct controls the expression of a novel chimeric reporter gene. The chimeric reported gene comprises the coding sequences from two separate genes, each of which producing a detectable gene product. In embodiments, one of the genes encodes a gene product that has an activity that is intrinsic (i.e., does not require the addition of substrate molecules or activator molecules), while the other gene encodes a protein that has an activity that can be detected at very low levels and also provides a high signal-to-noise ratio. In certain embodiments, the chimeric reporter gene comprises sequences encoding a green fluorescent protein (GFP), such as the enhanced green fluorescent protein (EGFP), or sufficient sequences to encode a portion of a GFP that can fluoresce. In certain embodiments, the chimeric reporter gene also comprises sequences encoding a luciferase protein, such as the Photinus luciferase, or sufficient sequences to encode a portion of a luciferase that can luminesce. The reporter construct of the invention allows those practicing the invention to perform clonal selection by detection of a signal due to the GFP. Fluorescence Activated Cell Sorting (FACS) or fluorescence microscopy can be used for detecting the signal, allowing for rapid single cell analysis and sorting. At the same time, a highly sensitive and reliable reporter signal is achieved by luciferase. Due to the intrinsic fluorescence of GFPs, the need to pre-load substrate molecules in order to detect cells that express the reporter gene is not required. Cell handling is therefore very simple, which makes the assay robust. Furthermore, cell viability after clonal selection is very high.

Accordingly, the present invention provides recombinant cells containing the reporter constructs of the invention. In addition to containing the reporter constructs, the recombinant cells can express at least one exogenous receptor, which can be, among other things, a G-protein coupled receptor, other membrane receptors, or an ion channel protein. That is, the recombinant cells can naturally express a G-protein coupled receptor or can contain non-homologous nucleic acids encoding G-protein coupled receptors. The recombinant cells of the invention express the reporter gene at high levels when the cells are exposed to substances that interact with a G-protein coupled receptor present on the cell surface, but do not express it to any appreciable level in the absence of a substance that interacts with a G-protein coupled receptor present on the cell surface.

The present invention also provides a method of making a recombinant cell. The method can include transforming, transfecting, or otherwise introducing a reporter construct of the invention into a suitable host cell to create a recombinant cell. The method can additionally include transforming, transfecting, or otherwise introducing a heterologous nucleic acid that expresses a cell surface receptor, such as a G-protein coupled receptor, into the host cell. The method can include preparing a stable recombinant cell that expresses heterologous proteins of interest from genes that are integrated into the host cell's genome. The method can also include procedures for performing fast clonal selection, for example by FACS or by ocular inspection of reporter activity (by changes in fluorescence, color, etc.). The method can also include preparing a transiently transformed recombinant cell that expresses at least one heterologous gene that is present in the recombinant as an extra-genomic element, such as a plasmid. The recombinant cells can be cell lines, and can be mammalian cells, insect cells, or other appropriate cells.

Thus, the present invention provides reporter constructs. The reporter constructs comprise a chimeric reporter gene that is operably linked to at least one responsive element. The reporter constructs are optimized by the practitioner for high level and stringent expression of the chimeric reporter gene in the chosen host cell and for the chosen cell surface receptor. For example, the number and spacing of the responsive elements present on the reporter construct can be optimized to provide high level expression only in the presence of a sufficient amount of the molecule to which the element is responsive. In this way, the reporter construct can help to minimize background signal and aid in the reliability and sensitivity of the overall system. In embodiments, the systems of the invention are used to detect substances that interact with target G-protein receptors. In embodiments, the invention uses a synthetic enhancer composed of multiple TPA (12-O-tetradecanoylphorbol- 13-acetate) responsive elements (TRE) fused to a minimal cytomegalovirus (CMV) promoter. The reporter constructs can be, but are not necessarily, present on a vector (e.g., plasmid).

In addition, the present invention provides methods of making the reporter constructs of the invention. The methods include molecular genetic techniques known to the skilled artisan to be useful for creating and modifying nucleic acids. The methods provide the reporter constructs of the invention, and are used to optimize directed expression of reporter genes in the assays of the invention.

The present invention further provides assays for detection of substances that interact with cell surface receptors. The assays can include exposing a recombinant cell of the invention to a sample containing at least one substance, and determining whether the sample activates expression of the recombinant reporter gene, thus indicating that at least one substance in the sample interacted with the cell surface receptor. The method can further include purifying, isolating, and/or identifying the substance that interacts with the cell surface receptor.

Accordingly, the present invention provides kits for performing the assay of the invention. The kits can, but do not necessarily, include all of the cells, constructs, reagents, and supplies necessary to detect binding of a substance to a cell surface receptor of interest. The kit can be used, for example, to identify drugs that modulate the activity of G-protein coupled receptor activated metabolic pathways.

### BRIEF DESCRIPTION OF THE DRAWINGS

This invention will be more fully described with reference to the drawings in which:

FIG. 1 depicts, generally, construction of a reporter construct of the invention by inclusion of varying numbers of AP-1 (TRE) motifs in the promotor region.

A. The first TRE was inserted, using PCR, directly in front of either the minimal CMV or minimal c-fos promotor.

B. The 9× TRE constructs were cloned by inserting the oligonucleotides O5-O8 in front of the first TRE.

C. The 9× TRE constructs were digested with Sac, and 4 TRE were removed to create the 5× TRE.

FIG. 2 schematically depicts a reporter construct of the invention. The plasmid pcFUSII was used to establish the stable HeLa reporter cell line, HF1. The construct contains an EGFP—firefly luciferase chimeric reporter gene, driven by a 9× TRE CMVₘᵢₙ promoter to ensure a sufficiently high signal to allow for detection of EGFP after stimulation of the HeLa host cells. The backbone from the pcDNA3 vector also contains a neomycin resistance cassette. The designation pA stands for the poly A tail. EGFP stands for the enhanced green fluorescent protein.

FIG. 3 shows the influence of the number of TRE, in combination with the minimal c-fos promotor or the minimal CMV promotor, on the induction of luciferase activity in two host cell lines.

A. HeLa cells.

B. CHO cells.

Cells electroporated for transient expression were stimulated with 100 nM PMA for 10 hours. Amplification was calculated as the ratio between the relative luminescence units (RLU) of stimulated and non-stimulated cells. Results are expressed as mean values+SEM of three to four independent transfection experiments each performed in triplicate; n.d.=not determined.

FIG. 4 show results from FACS analyses. HF1pBLTR cells were stimulated with 2×10⁻⁸ M leukotriene B4 and then sorted in a Becton-Dickinson FACS Vantage. Ten percent of the cells that expressed the highest EGFP level were gated and expanded.

A. Unstimulated cells.

B. Stimulated cells, where the arrow indicates the 10% portion of the cells that were gated and expanded.

FIG. 5 illustrates the response of endogenous ATP receptors present in the HeLa cells used to establish a reporter cell line, HF1, of the invention.

A. Dose-response curve following stimulation with varying concentrations of ATP for 16 hours. The calculated EC₅₀ value is 1.07×10⁻⁴ M. Shown are mean values from a typical experiment performed in quadruplicate. Error bars indicate± SEM.

B. Time-course of the TRE-mediated response of the HF1 reporter cells of the invention, grown in a 96-well format, following stimulation with 10⁻⁴ M ATP at the indicated time points, to induce and report a response mediated by the endogenous ATP receptors present on the target cells. Shown are mean values from one typical experiment performed in quadruplicate. Error bars indicate± SEM.

FIG. 6 shows model experiments in which reporter cells of the invention were tested with three types of receptors activated with ligands representing three widely different families of chemical mediators.

A. A monoamine (epinephrine) was the ligand.

B. A lipid mediator (LTB₄) was the ligand.

C. A peptide (having the sequence RANTES) was the ligand.

Dose-response curves are depicted for stimulation of reporter cells expressing the alpha adrenoceptor, Rα_{1b} (in A), the leukotriene B4 receptor, BLTR (in B), and the chemokine receptor, CCR5 (in C). Each receptor was stably expressed in the HF1 reporter cells of the invention and stimulated with their respective agonist. The values for the agonist concentration giving half maximum effect (EC₅₀) in these experiments were: for leukotriene B4 interacting with BLTR, 4.4×10⁻⁸ M; for epinephrine interacting with Rα_{1b}, 1.17×10⁻⁷ M; and for RANTES interacting with CCR5, 1.11×10⁻⁷ M. Shown are mean values from a typical experiment performed in quadruplicate. Error bars (mostly too small to be visible) indicate SEM.

FIG. 7 shows the results of experiments designed to test whether the levels of expression of reporter constructs of the invention can be altered with inhibitors. Reporter cells were treated with various compounds (indicated to the right) in a concentration of 1 μM each 30 minutes before agonist stimulation was started. A typical set of experiments performed in quadruplicate is shown. Error bars indicate± SEM. Statistical significance analysis was performed with Student's t-test.

FIG. 8 schematically and generally depicts an assay according to the invention.

FIG. 9 shows the results of reporter construct expression of pcFUSII in S2 insect cells upon treatment with various drugs that influence calcium release.

### DETAILED DESCRIPTION OF THE INVENTION

The present invention provides reporter systems for detecting substances that interact with cell surface receptors or ion channels. The reporter systems utilize recombinant cells expressing a cell surface receptor or ion channel of interest and a reporter gene whose expression is under the control of at least one molecule produced or otherwise made available as a result of interaction of the cell surface receptor or ion channel and another molecule (e.g., a ligand). The reporter systems of the invention are rapid, reliable, and simple to use. The reporter systems also provide a clonal selection method that for fast and efficient establishment of the best responding receptor specific reporter cell lines. The reporter constructs of the invention are functional in a variety of cell types and with a variety of cell surface receptors and ion channels, which is an advantage over constructs known in the art. The ability of the constructs to function in a variety of cell types is advantageous because several cell lines express endogenous receptors or ion channels that will make them unacceptable. Endogenous receptors or channels might interfere either by interacting with ligands shared by the recombinant test receptor or channel or, when using complex ligand mixtures, the endogenous receptors or channels might respond in concert with the target receptor or channel. As used hereinbelow, unless indicated otherwise, “receptor” is used generally to indicate both receptors and ion channels, and should only be interpreted as limited to receptors when an interpretation that includes “ion channels” would be inconsistent with the function of ion channels or with the application in general.

In a first aspect of the invention, nucleic acids comprising reporter constructs are provided. The nucleic acids can be any nucleic acid that encodes a chimeric gene according to the invention and that is capable of being expressed in a target cell. Thus, the nucleic acids of the invention can be RNA or DNA, double-stranded or single-stranded, linear or closed circular, concatameric, and/or supercoiled.

In embodiments, the nucleic acids of the invention comprise constructs and elements known to the skilled artisan. For example, the nucleic acids can be expression vectors or shuttle vectors. Examples include, but are not limited to, plasmids; viruses and viral nucleic acids, including phages and phage nucleic acids; cosmids; phagemids; and artificial chromosomes, including Bacterial Artificial Chromosomes (BACs) and Yeast Artificial Chromosomes (YACs). The nucleic acids can be provided as naked nucleic acid or can be provided as part of a mixture or complex with other molecules that aid in targeting and delivering nucleic acids to host cells. For example, the nucleic acids can be provided in a composition that includes liposomes, cell- or tissue-specific antibodies, or cell- or tissue-specific ligands to increase the uptake of the nucleic acids into the host cells.

The reporter constructs of the invention include a chimeric reporter gene that is operably linked to at least one transcription control element. Transcription control elements constitute parts of promoters or enhancers where at least one protein or protein complex can bind. Thus, in embodiments, the chimeric reporter gene is operably linked to a promoter and/or at least one enhancer sequence. A promoter or enhancer, and thus a transcriptional control element, is operably linked to a coding sequence (for example, a chimeric reporter gene of the invention) if it participates in regulation of transcription of the coding sequence. Various transcription control elements are known to those of skill in the art, and all are applicable to the present invention. Examples of transcription control elements include, but are not limited to, cAMP responsive elements (CRE) and TPA responsive elements (TRE; AP-1), or any other transcription control element that is involved in gene transactivation upon stimulation of surface receptors. Other transcription control elements are disclosed in U.S. Pat. Nos. 5,401,629 and 5,435,128 to Harpold et al. and U.S. Pat. No. 5,854,004 to Czernilofsky et al., the disclosures of which are incorporated herein in their entireties by reference.

In embodiments, the transcription control element is responsive to intracellular signals that can be generated, either directly or ultimately, as a result of binding of a cell surface receptor to a ligand. For example, the transcription control element can be responsive to cyclic adenosine monophosphate (cAMP) or phorbol-12-myristat-13-acetate (TPA).

The reporter constructs include at least one chimeric reporter gene whose expression is controlled by at least one transcription control element. Expression can be up-regulated or down-regulated in response to an intracellular signalling molecule. Preferably, in the absence of the intracellular signalling molecule, there is little or no detectable expression of the chimeric reporter gene. In embodiments, multiple transcription control elements are operably linked to a single chimeric reporter gene. In these embodiments, the reporter constructs are optimized for high level and stringent expression of the chimeric reporter gene in the chosen host cell. For example, the number and spacing of the transcription control elements present on the construct are optimized to provide high level expression only in the presence of a sufficient amount of the molecule to which the element is responsive. By including multiple copies of a single control element, more than one type of control element, or a combination of the two, the reporter constructs of the present invention can be optimized to minimize background signal and aid in the reliability and sensitivity of the overall system. Examples of transcriptional control elements are AP-1, CRE, and NFAT.

A minimal promoter, though not in itself necessary, constitutes the smallest fragment of a promoter that still has the capacity to direct transcription. The above-mentioned two components (i.e., at least one transcription control element and a minimal promoter) are, in the present context, defined as a “reporter control element”. In embodiments, more than one type of reporter control element is operably linked to a single chimeric reporter gene.

The reporter constructs of the invention comprise at least one chimeric reporter gene (also referred to herein as a reporter fusion gene). The chimeric reporter gene comprises the coding sequences for at least two proteins, or functional portions (i.e., fragments) thereof. Suitable reporter genes are those genes whose expression products can be monitored without the need to lyse or otherwise destroy or diminish the viability of the cell in which they are expressed. A “function portion” is a sufficient amount of a coding sequence to encode a protein or polypeptide that has an activity that can be monitored without the need to lyse or otherwise destroy or diminish the viability of the cell in which it is expressed. In preferred embodiments, the activity of the fragment is the same activity as that of the full-length protein from which it is derived. Because the reporter proteins expressed from the reporter construct are easily detectable, identification of functional portions of the proteins is a straightforward matter that does not require undue or excessive experimentation.

Suitable reporter genes are known in the art, and include luciferase, antibiotic resistance, heavy metal resistance, and other genes whose expression can be detected by luminescence, fluorescence, a chemical reaction that results in a color change of a reagent, or some detectable phenotypic change in the cell into which the gene is introduced. Examples of reporter genes include, but are not limited to, firefly luciferase, bacterial luciferase, Renilla luciferase, Photinus luciferase, green fluorescent protein (GFP), the enhanced green fluorescent protein (EGFP), chloramphenicol acetyl transferase (CAT), alkaline phosphatase, and β-galactosidase.

The choice of reporter genes used to create the chimeric reporter gene in the reporter construct can be based on the preference of the worker skilled in the art. Standard molecular biology techniques, well-known and widely practiced by those of skill in the art, can be used to create the chimeric reporter gene. For example, restriction endonuclease cleavage and religation can be used to fuse the coding regions of two reporter genes to create a chimeric reporter gene. Where necessary, oligo-directed engineering of restriction endonuclease cleavage sites can be used to ensure cleavage at desired points in the reporter genes, for example to maintain the proper reading frame in the chimeric reporter gene.

The reporter constructs of the invention can further comprise selection markers, including, but not limited to, antibiotic resistance genes and heavy metal resistance genes. Selection markers are well known to those of skill in the art and thus need not be listed in detail here. The selection markers can be useful in preparing large quantities of the construct for use in the assays of the invention, or can be used, for example, as a selection marker for recombinant cells of the invention and for maintenance of pure cultures of the recombinant cells. In addition, the reporter constructs of the invention can comprise an origin of replication to enhance replication and maintenance of the construct in the host.

The reporter constructs of the invention permit those practicing the invention to create recombinant cells that express a desired level of a reporter gene in response to activation (or repression) via the reporter control element(s). For example, the reporter constructs enable the practitioner to maximize the level of expression of the chimeric reporter gene upon induction by a pre-selected intracellular signalling molecule, such as one known to be linked to a chosen receptor and/or signal transduction pathway. In embodiments of the invention, an optimized reporter construct is used in the construction of heptahelix receptor-based reporter cell lines. In these embodiments, the promoter comprises multiple TRE motifs fused to a minimal promoter. In embodiments, the reporter construct is pGL3-AP1×1 FOS. In embodiments, the reporter construct is pGL3.AP1×9 FOS. In embodiments, the reporter construct is pGL3-AP1×1 CMV. In embodiments, the construct comprises nine TRE motifs, such as in the reporter construct pGL3.AP1×9 CMV. In embodiments, the reporter construct is pcFUSII. In yet other embodiments, the reporter construct is pcFUS3. Exemplary reporter constructs are described in more detail in the Examples that follow, and in the Figures.

In another aspect, the present invention provides methods of making the reporter constructs of the invention. The methods include molecular genetic techniques known to the skilled artisan to be useful for creating and modifying nucleic acids. The methods provide the reporter constructs of the invention, and are used to optimize directed expression of reporter genes in the assays of the invention. In general, commonly available nucleic acid molecules, such as vectors, are modified by addition of at least one reporter gene and at least one transcription control element such that production of a detectable reporter protein is either enhanced or reduced as the result of binding of a signalling molecule (e.g., a transcription factor) to the transcription control element. Multiple copies of a single transcription control element can be operably linked to a single reporter gene. In addition, multiple types of reporter control elements can be operably linked to a single reporter gene. Furthermore, a mixture of different numbers and types of control elements can be operably linked to a single reporter gene. The selection of reporter control element(s), as well as the number of copies of each, should be optimized to provide the highest level of expression of the reporter gene in the host cell. In addition, it is preferable that, under conditions where expression is not desired, the level of expression is at, near, or below, the level of detection. The reporter construct is optimal for various cell types, but the total signal and the signal-to-noise background ratio may differ for the individual cell type containing the construct of the invention. The signal-to-noise ratio may be improved by introducing into the cells one or more recombinant genes coding for necessary components in the signal transduction pathway being utilized. Optimizing the number of reporter control elements for the chosen cell is a routine, straightforward matter that can be accomplished rapidly by those of skill in the art.

In another aspect, the present invention provides recombinant cells. The recombinant cells of the invention contain the reporter constructs of the invention. The recombinant cells can express at least one reporter gene present on the reporter construct. Expression of the reporter gene is regulated by at least one transcription control element that is responsive, ultimately, to interaction of a cell surface receptor and a ligand. Expression of the reporter gene can either be up-regulated or down-regulated in response to interaction between the cell surface receptor and the ligand. In embodiments, expression of the reporter gene is up-regulated in response to the interaction of the cell surface receptor and the ligand. The ligand can be any substance or microorganism that interacts with the cell surface receptor, including, but not limited to, drugs, prodrugs, and viruses. The substance, or ligand, can be organic or inorganic.

In embodiments, the recombinant cells of the invention express the reporter gene at high levels when the cells are exposed to a substance (e.g., a ligand) that interacts with a cell surface receptor, but do not express it to any appreciable level in the absence of a substance that interacts with the cell surface receptor. In these embodiments, the cell surface receptor can be, but is not limited to, a G-protein coupled receptor, a tyrosine kinase-type receptor, or an ion channel receptor.

Thus, in addition to containing the reporter constructs, the recombinant cells express at least one cell surface receptor. The cell surface receptor can be expressed from an endogenous gene (i.e., a gene that was not introduced into the cell using molecular biology technology) or recombinantly (i.e., as a result of introduction of a gene into the host cell by molecular biology technology). In embodiments, expression of a gene naturally present in the genome of the host cell can be augmented by introduction, via molecular biology technology, additional copies of the gene, resulting in a recombinant cell. Thus, the cell surface receptor gene can be present in the recombinant cell in single, double, or multiple copies, and can exist genomically (i.e., in the host chromosome), extrachromosomally, or both. In embodiments, the cell surface receptor is expressed from a gene present on the reporter gene construct. In embodiments, the cell surface receptor is expressed from a gene present on a construct that is separate from the reporter gene construct. In embodiments, expression of the cell surface receptor is unregulated (i.e., it is constitutively expressed), while in other embodiments, expression of the cell surface receptor is regulated.

In embodiments, the cell surface receptor is a G-protein coupled receptor. In embodiments, the cell surface receptor is an ion channel receptor. In embodiments, the cell surface receptor is a tyrosine kinase-type receptor. Preferably, the receptor, or a majority of the receptor that is expressed, is localized to the cell surface. Examples of G-protein coupled receptors include, but are not limited to, the leukotriene B4 receptor (BLTR), the chemokine receptors CCR5 and CXCR4, the alpha1b adrenoceptor, and the C5a receptor.

In embodiments, especially embodiments where a non-endogenous cell surface receptor is expressed, the recombinant cell does not naturally transfer the signal produced by the cell surface receptor to the transcription control element because one or more members of the signalling pathway are absent or function poorly. In these embodiments, the absent or poorly functioning pathway member(s) can be provided to the cell as a recombinant “helper” protein(s). The recombinant helper protein(s) can be expressed from the reporter construct or from separate expression vector(s). In addition, they can be expressed from vectors that have integrated into the host cell genome.

In a further aspect, the present invention provides a method of making a recombinant cell. The method can include transforming, transfecting, or otherwise introducing a reporter construct of the invention into a suitable host cell to create a recombinant cell. Techniques for transforming, transfecting, or otherwise introducing nucleic acids, viruses, etc. into eukaryotic cells are known to those of skill in the art. Any suitable technique can be used so long as it does not result in unacceptable alteration of the reporter construct, other vectors (when used to co-express other genes), or the host cell. Unacceptable alterations include alterations that render the nucleic acids and cells unsuitable for their intended purposes.

In embodiments, the method additionally includes transforming, transfecting, or otherwise introducing a heterologous nucleic acid that encodes a cell surface receptor, such as a G-protein coupled receptor, into the host cell. Introduction of the heterologous nucleic acid encoding the cell surface receptor can be accomplished before, at the same time, or preferably after, introduction of the reporter construct into the host cell. In embodiments, the gene encoding the cell surface receptor is present on the reporter construct. In other embodiments, the gene encoding the cell surface receptor is present on a separate nucleic acid construct.

The method can include preparing a stable recombinant cell that expresses heterologous proteins of interest from genes that are integrated into the host cell's genome. Alternatively, the method can include preparing a stable recombinant cell that expresses heterologous proteins of interest from genes that are not integrated into the host cell's genome (e.g., from genes present on an Epstein-Barr viral vector). The method can also include preparing a transiently transformed recombinant cell that expresses at least one heterologous gene that is present in the recombinant as an extra-genomic element, such as a plasmid. The invention provides a method for quick selection of the best expressing recombinant clones. Techniques for preparation of stably- and transiently-transfected cells are known to those of skill in the art. Generally, cells constituting the system are the progeny of a single ancestral transformant. Recombinant expression systems as defined herein will express heterologous protein upon induction of the regulatory elements linked to the DNA sequence or synthetic gene to be expressed.

The recombinant cells can be cell lines, and can be mammalian or non-mammalian. In embodiments, mammalian cell surface receptors are recombinantly expressed in insect cells. In general, because many mammalian transcription control elements are active in other eukaryotic cells, such as insect (e.g., Spodoptera frugiperda ovarian (Sf9, Sf21) cells) and other non-mammalian (e.g., yeast, nematode) cells, it is possible to use mammalian reporter constructs and recombinant cell receptors in such cells. For example, AP-1 elements from mammalian cells, which are responsive to, among other things, intracellular calcium levels, can also function in insect cells if a receptor system is in place that mobilizes calcium.

An additional aspect of the invention is an assay for detection of substances that interact with cell surface receptors. Broadly, the principle of the assay of the invention is depicted in FIG. 8. In general, the assay includes exposing a recombinant cell of the invention (including a culture of the cell) to a sample and determining whether expression of a reporter gene present on the reporter construct is altered. Alteration (i.e., up- or down-regulation) indicates that the sample contains at least one substance that can interact with a receptor present on the surface of the recombinant cell. Alteration in reporter gene expression is easily assayed using reagents, protocols, and equipment widely known and available to those in the art. For example, many commercial vendors sell systems for expression and detection of a signal from luciferase, CAT, β-galactosidase, and alkaline phosphatase. Other systems, though not commercially available, are known to the skilled artisan, and can be used in accordance with the present invention.

The assay can be performed with intact or lysed cells in any suitable volume of culture media. In preferred embodiments, the assay is performed in microtiter plates, such as a 96 well or 384 well plate. In these embodiments, some or all of the wells of the microtiter plate contain a culture of the recombinant cell of the invention. Each culture can be exposed to a sample containing the same or different substances. Thus, the same microtiter plate can be used to assay multiple substances for their ability to interact with a selected cell surface receptor. In addition, a sample can be assayed multiple times using multiple wells in a single microtiter plate to verify its activity or lack thereof. In all instances, the signal is related to that obtained in control cells lacking a recombinant test receptor.

The assay of the invention can be a high-throughput assay that can be used to screen large numbers of substances or mixtures of substances that interact with a chosen cell surface receptor. For example, in embodiments of the invention, a recombinant cell expressing a cell surface receptor of the superfamily of G-protein coupled receptors interacts with a substance, which causes the receptor to generate a signal that subsequently activates the reporter gene on the reporter construct. The level of expression of the reporter gene product is monitored by the appropriate techniques (fluorescence, luminescence, color change).

In embodiments, the method of assaying for substances that interact with cell surface receptors further includes purifying, isolating, and/or identifying the substance that interacts with the cell surface receptor. In these embodiments, techniques known to the skilled artisan can be used to purify and/or isolate the substance(s). Such techniques include, but are not necessarily limited to, precipitation, filtration (including size-exclusion chromatography), liquid chromatography, paper chromatography, centrifugation, affinity chromatography, and solvent extraction.

The reporter system of the invention can include clonal selection of the recombinant cells. Thus, in embodiments, the method of making a cell according to the invention includes clonal selection of the cells. Accordingly, in embodiments, the assay of the invention includes, prior to screening for molecules that affect the activity of a cell surface receptor, clonal selection to obtain efficiently expressing cells. Clonal selection can be carried out using any techniques known to those of skill in the art. For example, it can be carried out using fluorescent analytical cell sorting (FACS), during illumination (activation) with UV light in a low-power operation microscope. Thus, the present assay avoids much of the time and labor required in the assays known in the art. The present system permits identification of well-responding cells in a fraction of the time that is necessary in other assays. As a consequence, the signal-to-noise ratio of the present assay is higher than other assays.

Clonal selection can be used advantageously in the construction of reporter cell lines and in practice of the assay of the invention. Often, the sensitivity of the final cell line can be substantially increased. The presence of a chimeric reporter gene in the construct according to the invention also allows for clonal selection by FACS or by ocular identification of the colonies with fluorescence microscopy. Thus, in an embodiment, a construct having a chimeric reporter gene that comprises EGFP fused in frame to Photinus luciferase is provided.

In another aspect of the invention, kits are provided. In embodiments, the kits are used to perform the assay of the invention (i.e., to identify samples that contain substances that interact with a specific cell surface receptor, or to detect such substances). The kit can, but does not necessarily, include all of the cells, constructs, reagents, and supplies necessary to detect binding of a substance to a cell surface receptor of interest. The kit can be used, for example, to identify drugs that modulate the activity of G-protein coupled receptor activated metabolic pathways. It can also be used, for example, to detect proteins or small molecules that interact with ion channels.

### EXAMPLES

The invention will now be further described with reference to examples of embodiments of the invention. The following examples are meant to more fully illustrate certain embodiments of the invention and are not to be construed as limiting the scope of the invention.

### Example 1

### Construction of a Reporter Plasmid

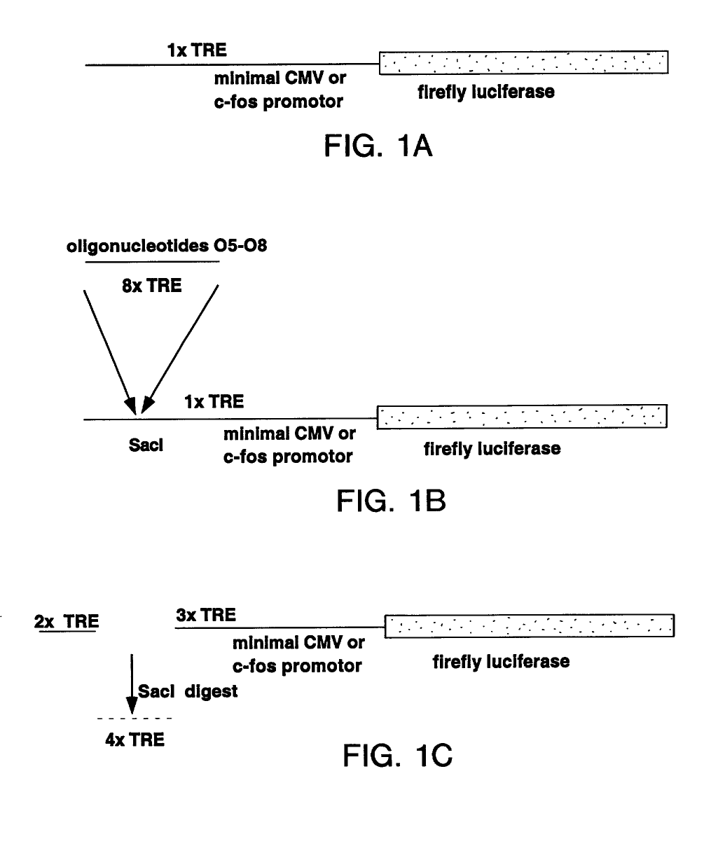

Construction of a reporter plasmid according to the invention is depicted generally in FIG. 1. In particular, the plasmid, pGL3 basic (Promega), was used as a backbone for the reporter construct according to the invention. Primers and oligonucleotides used in the invention are shown in Table 1, in which consensus TRE motifs are shown in bold type, and restriction endonuclease sites are underlined.

TABLE 1

| Name of | | |
|---|---|---|
| oligonucleotide | Sequence (5′ to 3′) | |
| P1 Apx1 CMV | GCAGATCTTCATGAGTCAGACAGGCGTGTACGG | (SEQ ID NO:1) |
| upper | | |
| P2 CMV lower | AGGAAGCTTCGGTCCCGGTG | (SEQ ID NO:2) |
| P3 Apx1 FOS upper | TCGAGCTCCATGAGTCAGACACTCATTCAT | (SEQ ID NO:3) |
| P4 FOS lower | ACATAAGCTTGGCGGTTAGGCAAAGCC | (SEQ ID NO:4) |
| O5 3xAP sense | ATGAGTCAGAGCTCAATGAGTCAGATGAGTCAGCT | (SEQ ID NO:5) |
| O6 3xAp antisense | GACTCATCTGACTCATTGAGCTCTGACTCATGGCT | (SEQ ID NO:6) |
| O7 APx1 sense | CTTGACGTCAAGCATGAGTCAGACAGAGCTCGTAGCC | (SEQ ID NO:7) |
| O8 APx1 antisense | ACGAGCTCTGTCTGACTCATGCTTGACGTCAAGGGCC | (SEQ ID NO:8) |
| P5 EGFP upper | TCCAAGCTTCGCCACCATGGTGAG | (SEQ ID NO:9) |
| P6 EGFP lower | GCGCCATGGTCATGAACTTGTACAGCTCGTCC | (SEQ ID NO:10) |

The minimal CMV promoter was amplified with primers P1 and P2, while the c-fos promoter was amplified by PCR using primers P3 and P4 and pc-FOS (ATCC 41042) as a template. The upper primer sequences contained one TRE each. This TRE was inserted at −54 position relative to the transcription start (minimal c-fos promoter) and 51 position (minimal CMV promoter). The PCR fragments were digested with BglII, HindIII (minimal CMV promoter), and SacI, HindIII (minimal c-fos promoter), respectively. The fragments were inserted in the appropriately digested vector. This resulted in the plasmids, pGL3-APx1 FOS and pGL3-APx1CMV, respectively. The plasmids were linearized with SacI and the 8× TRE box (corresponding to oligonucleotides O5- O8) was inserted. This resulted in the plasmids pGL3-APx9 FOS and pGL3-APx9 CMV. By inserting the 8× TRE box into the vector, the SacI site of the vector was destroyed and new SacI sites were introduced with oligonucleotides O5 and O6. In order to obtain the 5× TRE constructs, the SacI fragment containing 4× TRE was removed and the plasmids re-ligated (FIG. 1). The primers and oligonucleotides were designed by the inventors and custom-synthesized at Gibco BRL.

### Example 2

### Construction of a Chimeric Reporter Gene

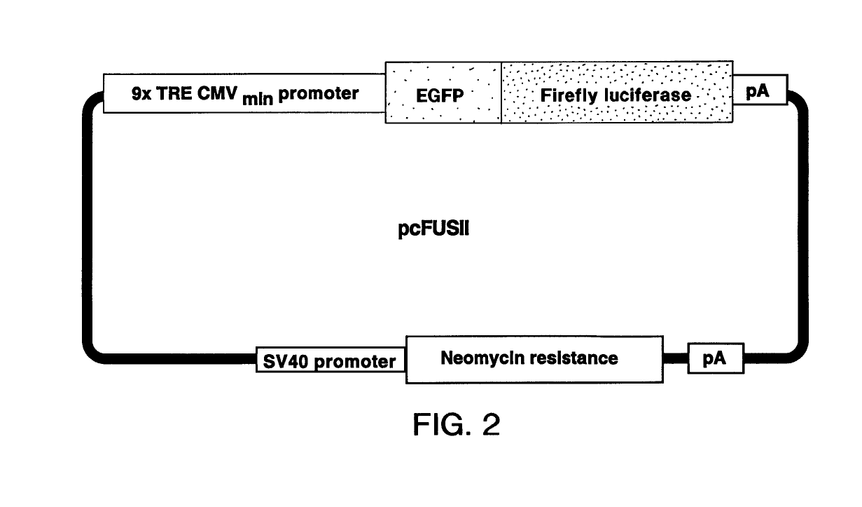

The plasmid, pEGFP-1 (Clontech), was used as template in a PCR reaction with the primers P5 and P6 to amplify the enhanced green fluorescent protein (EGFP). The product was cut with NcoI and BspHI and inserted in front of the luciferase gene into the NcoI site of pGL3-APx9 CMV to get the plasmid pFUSII. The stop codon at the end of EGFP was thereby removed, giving rise to a fusion protein between EGFP and firefly luciferase. The plasmid, pFUSII, was digested with BamHI and KpnI, and the fragment containing the complete reporter construct was ligated into the backbone of the pcDNA3 plasmid between the BglII/KpnI sites, thereby replacing the CMV promoter in pcDNA3. The resulting plasmid, pcFUSII, contains the reporter construct and a neomycin resistance cassette (FIG. 2; SEQ ID NO: 11).

### Example 3

### Construction of Receptor Plasmids

Three prototypic receptors were tested in the reporter system. All receptor ORFs were inserted into the pIRESpuro vector (Clontech) by standard techniques. The alpha adrenergic receptor, Rα_{1b} cDNA was a kind gift from Dr. Robert Lefkowitz (see Lomasney et al., Journal of Biological Chemistry, 266:6365-6369, 1991), the chemokine receptor, CCR5, was cloned by the inventors from a human monocyte cDNA by PCR and sequenced, and the cDNA encoding the human leukotriene B4 receptor, BLTR, had earlier been cloned in our laboratory (Owman et al., Genomics, 37:187-194, 1996; Owmian et al., Biochemical and Biophysical Research Communications, 240:162-166, 1997).

### Example 4

### Selection of Reporter Cell Lines

Cell Culture

HeLa and CHO cells were grown in Dulbecco's modified Eagle's medium (DMEM) with Glutamax I, supplemented with 10% fetal bovine serum, 0.5% streptomycin and penicillin at 37° C. and 7% CO₂.

Transfection

HeLa and CHO cells were electroporated essentially as described by methods known to the art (see Rols et al., Nucleic Acids Research, 22:540, 1994). Briefly, by using a ElectroSquarePorator T820 (Genetronics; BTX), 5×10⁶ cells were pulsed in electroporation buffer (10 mM phosphate buffer, 250 mM sucrose, 1 mM MgCl₂, pH 7.2) in a 4-mm gap cuvette 15 times for 3 msec with 150 V. Before pulsing, cells were mixed with 6-10 micrograms of plasmid and incubated 10 min on ice. The cells were kept for 10 min at 37° C. after electroporation. In stimulation experiments following transient expression, cells from one transfection were split into 6 wells of a 24-well plate.

Cells grown in 15 cm diameter dishes were electroporated with 9 micrograms of linearized plasmid to establish stable HeLa cell lines. After 2 days, the medium was supplemented with 1 μg/ml G418 or 1 μg/ml puromycin, respectively. The medium was renewed every second day for two weeks.

Ocular Selection Procedure

After approximately 2 weeks, 50 to 200 colonies per plate had grown up. For the selection of HF reporter cell lines, the medium was supplemented with 100 nM PMA for about 16 h. Colonies were checked under UV light using an Olympus inverted microscope with appropriate fluorescence filters. Green colonies were picked with a pipette, expanded, and tested as reporter cell lines. The different receptors were stably transfected by electroporation and selected for puromycin (1 μg/ml) resistant clones. Clones were picked, expanded, and analyzed for their capability to activate the reporter gene after receptor stimulation with the appropriate agonist. This procedure gave rise to HF1pBLTR cells, HF1pRα_{1b} cells, and HF1pCCR5 cells.

FACS Selection Procedure

A FACS Vantage machine from Becton-Dickinson was used. HF1 cells and HF1pBLTR cells were grown in 6-well plates. Cells were stimulated with 3×10⁻⁴ M ATP or 2×10 ⁻⁸ M LTB₄, respectively, 16 h prior FACS. The cells were trypsinized, washed three times with 10 ml PBS without magnesium and calcium, and suspended in PBS containing 1 mM EDTA at 500,000 cells per ml 1h before FACS. From HF1 cells two pools were then sorted out: 100,000 cells representing 20% of the population and 40,000 cells representing 5% of the best responding cells, respectively. HF1pBLTR cells (100,000 cells representing 10% of the best cells) were sorted out. The best responding cells are defined as cells containing most EGFP. These pools were grown up, cultured for 2 weeks in parallel with the mother cell lines, and then used in ligand stimulation experiments as described.

### Example 5

### Luciferase Assay and Stimulation Experiments

Luciferase Assay

Cells transiently transfected with the various promoter constructs were stimulated 24 h after transfection with 100 nM PMA for 10 h in 24-well plates. The medium was then removed, cells were washed once with PBS, and 100 microliters reporter lysis buffer (Promega) were added per well. The plates were stored until analysis, usually overnight at −20° C. Luciferase assays were performed with Luciferase Assay Kit (Biothema, Sweden) according to the manufacturer's instruction. Transiently transfected cells were analyzed in a Turner TD-20e luminometer. Luciferase assay for stably transfected clones was carried out with a BMG Lumistar luminometer in 96-well plate format. White, clear-bottom plates of tissue culture quality (Costar) were used. Approximately 10,000- 20,000 cells were grown per well in 90 μl medium. After 3 days, ligands were added in 10 μl PBS and incubated for further 16 h. The medium was removed and cell lysis buffer added. Plates were stored at −70° C. until further analysis. All experiments were performed two to four times in quadruplicate.

Fluorescence Analysis in 96-Well Plate Reader

The fluorescence measurements were performed in a BMG Fluostar fluorometer in black plates with clear bottom (Costar). Cells cultured and stimulated as above were assayed in 100 μl PBS. After fluorescence measurement, the PBS was removed and the luciferase activity determined as described above.

Inhibitors

HF1, HF1pBLTR, and HF1pRα1b cells were grown in 96-well plates as described. The respective inhibitor was added to the cells at 1 μM concentration 30 min before stimulation with the respective agonist. Luciferase assay was performed after 16 h.

Calculations

All calculations were performed in the GraphPad prism computer program.

### Example 6

### Modification of pcFUSII for Optimization in Insect Cell Lines

Plasmid pcFUSII was modified for use in insect cells by replacing the SV40 promoter of the neomycin resistance gene with the baculovirus IE-1 promoter from the plasmid pIE1-3 (Novagen). This was done by digesting pcFUSII with EcoRI/AflII and blunting the AflII site. An EcoRI/SmaI fragment from pIE1-3, containing the IE1 promoter, was then ligated into this site, thus resulting in the plasmid pcFUSII-IE (SEQ ID NO:12).

### Example 7

### G-Protein Chimeric Construction

Because not all of the native insect G-proteins are able to efficiently transduce the signal from a mammalian receptor, some of the reporter systems that are based on insect cells were also made to contain a G-protein expression unit. This unit is composed of a constitutive, i.e., unregulated, promoter that controls the transcription of either a mammalian G_{α} or a chimeric G_{α} subunit. This expression unit is then inserted into the basic reporter construction (FIG. 2). The chimeric G-protein is based on the gene of an insect G_{α} subunit (dG G_{α}q3), where the last five amino acids have been replaced with the last five amino acids of either the human G G_{α}i2 or G G_{α}16 subunit. This was accomplished by the polymerase chain reaction (PCR) using the primers described in Table 2 and the gene for dGq-3 (Talluri S., et al., PNAS, 92:11475-11479, 1995) as a template.

TABLE 2

| Name | Sequence | |
|---|---|---|
| dGqU | TAT GCG GCC GCT TAG CAT GGA GTG CTG | (SEQ ID NO:13) |
| dGq-i2L | CTA GAT CTC AGA AGA GGC CGC AGT CCT TAA GGT TCG ATT G | (SEQ ID NO:14) |
| dGq-16L | CTA GAT CTC ACA GCA GGT TGA TCT CCT TAA GGT TCG ATT G | (SEQ ID NO:15) |

The template was amplified for 10 cycles (20 sec at 95° C., 30 sec at 55° C. and 2 min at 72° C.) with Pwo polymerase and 2 mM MgSO₄. The product was digested with BglII/NotI and was subsequently ligated into pIE1-3. The resulting plasmid was digested with EcoRI and HindIII and blunted. The expression cassette, containing the IE1 promoter and the chimeric G-protein, was purified and ligated into a filled-in BsmI site of the pcFUSII-IE reporter vector.

### Example 8

### Inhibition of Reporter Constructs

The ability of the reporter constructs of the invention to be inhibited by chosen inhibitors was tested. FIG. 7 shows the results of these experiments. The graphs show a reporter cell line of the invention, HF1, expressing no recombinant receptor, HF1pRα_{1b}reporter cells expressing the alpha-adrenergic test receptor, and HF1pBLTR reporter cells expressing the leukotriene B4 test receptor. Each cell was exposed to a) agonist only (control), b) UO126, c) DHBP, or d) GF109203X, as described below.

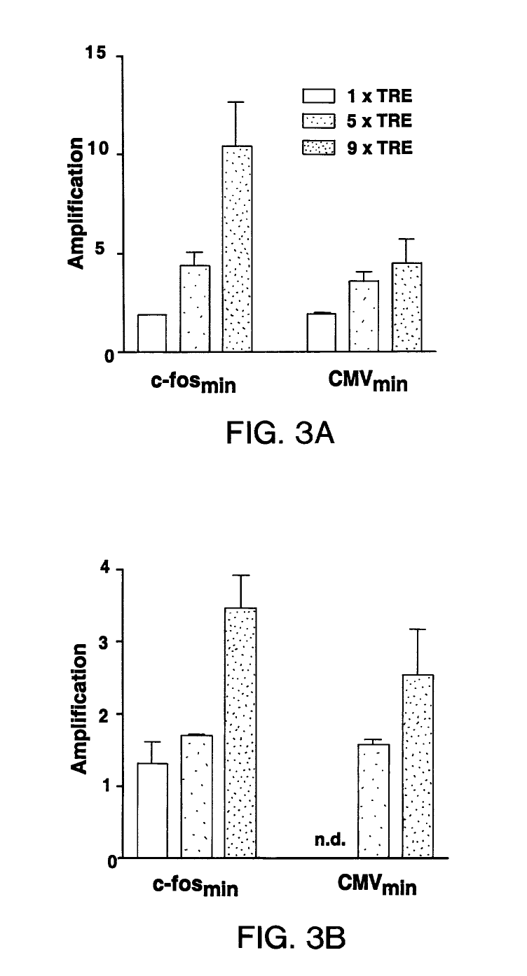

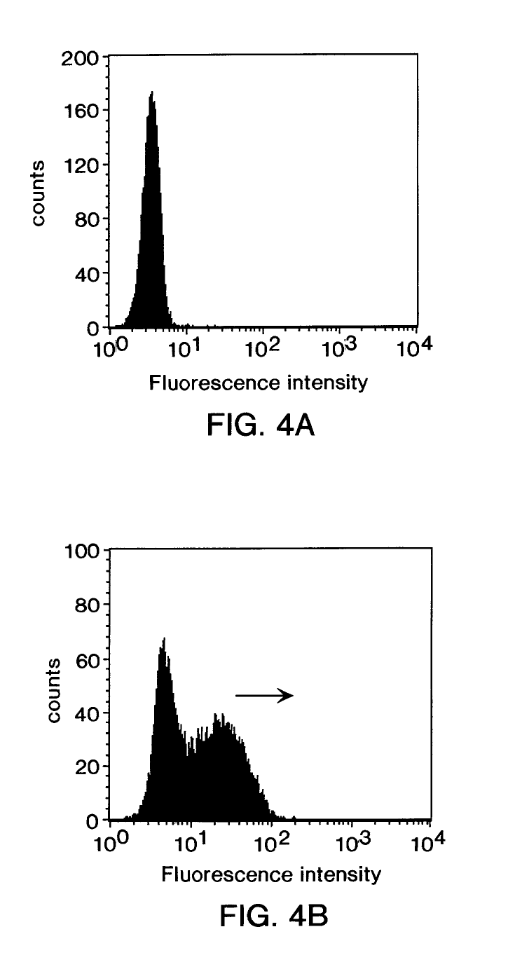

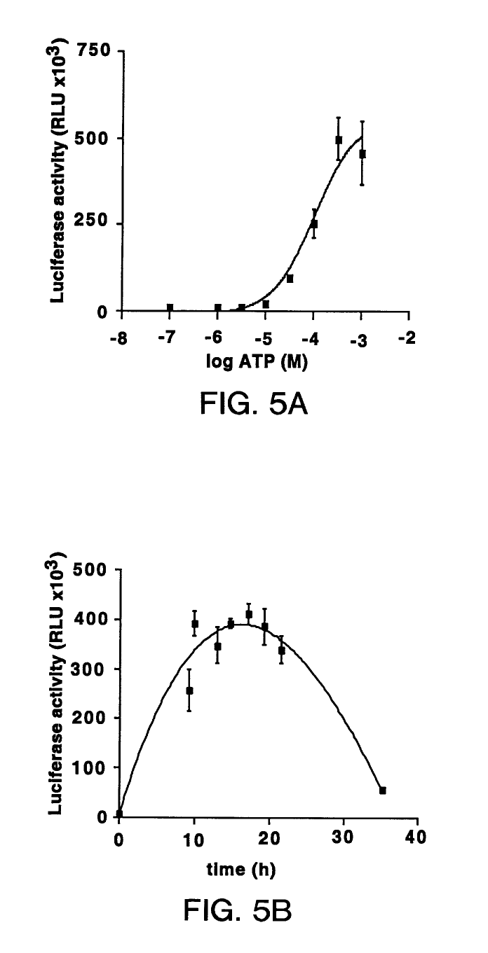

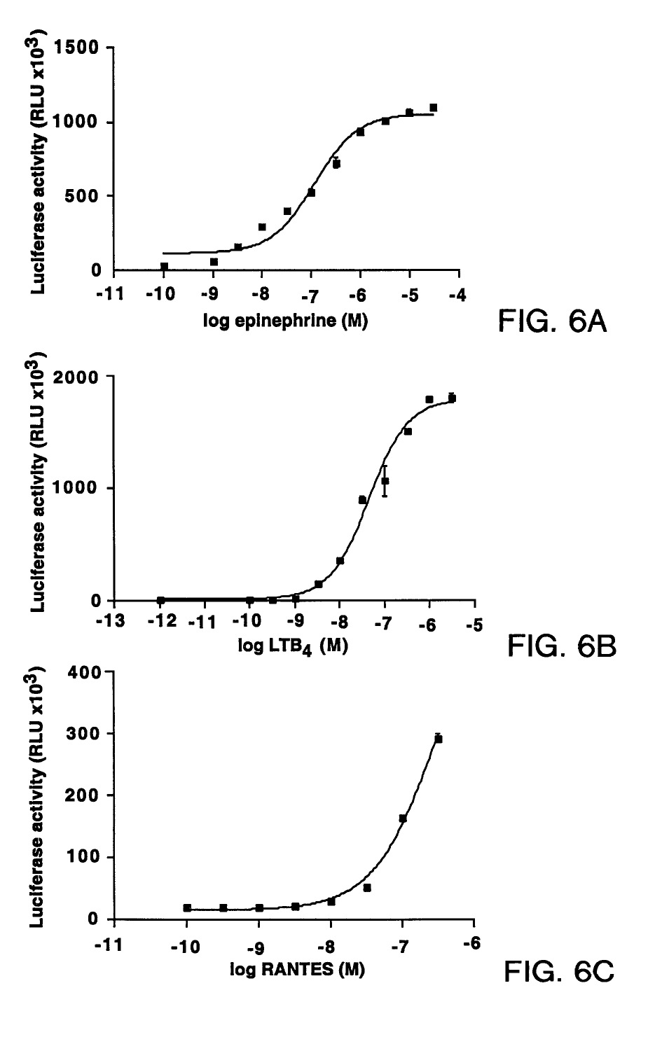

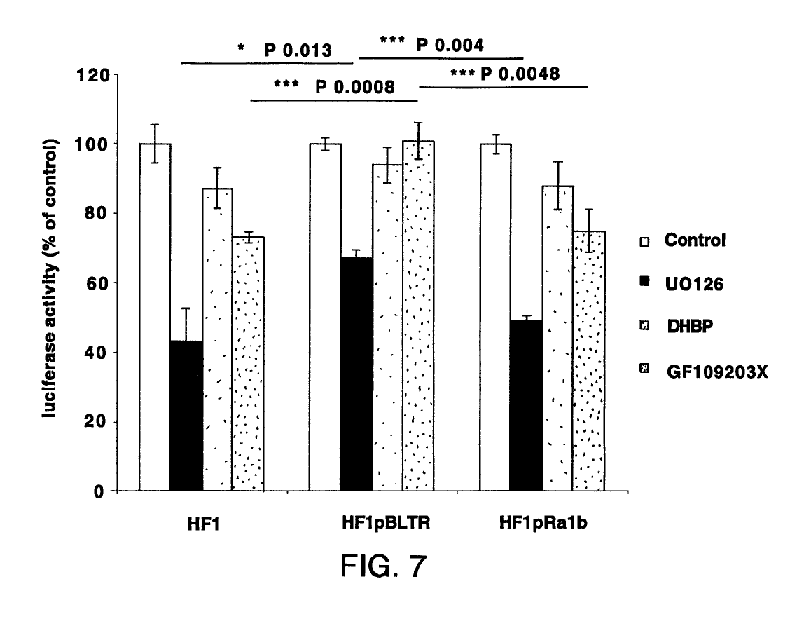

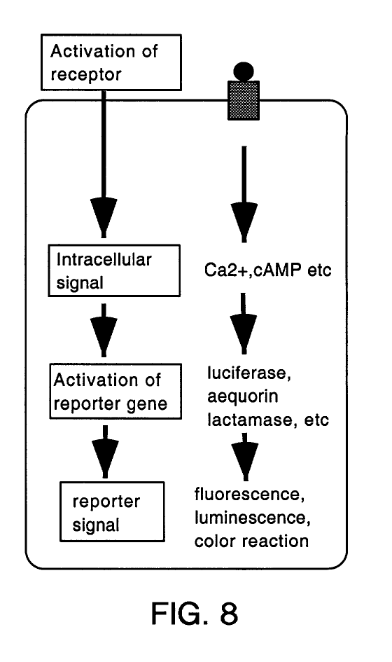

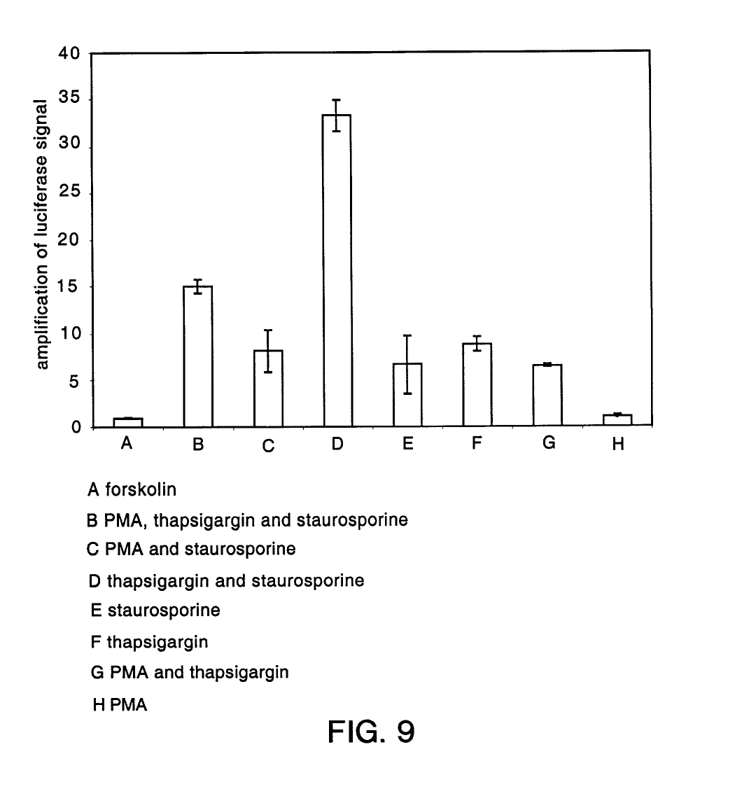

The HF1 cells were first stimulated with ATP (at the maximum concentration illustrated in FIG. 3) to activate the endogenous ATP receptors, then the cells expressing recombinant receptors (HF1pBLTR and HF1pRa1b) were stimulated with their respective agonist (at the maximum concentration illustrated in FIG. 4A and 4B, respectively). The reporter cells were treated with the compounds (indicated to the right) in a concentration of 1 μM each at 30 min before the agonist stimulation was started, in order to inhibit different signal transduction pathways. Luciferase activity in cells treated with agonist only (control) was taken as 100%. A typical set of experiments performed in quadruplicate is shown. Error bars indicate± SEM. Statistical significance analysis was performed with Student's t-test.

The results indicate that various compounds can be used to inhibit the expression of reporter constructs of the invention. This result further indicates that the systems of the invention can not only be used to identify compounds or molecules that positively affect the level of signal generated by the reporter constructs and reporter cells of the invention, but that the system can be used to identify compounds or molecules that negatively affect the level of signal. Furthermore, this result shows that compounds can be added to the system to regulate the intensity of signals generated by the reporter constructs and cells. That is, inhibitor compounds or molecules can be added to the assay of the invention, in amounts chosen by the artisan practicing the invention, to adjust the intensity of the signal, such that a desired level of signal is produced by the assay.

### Example 9

### Reporter Systems Based on Insect Cell Lines

Test of the Reporter Construct pcFUSII in Insect Cells

In order to test if the reporter construct pcFUSII is activated in insect cells upon calcium mobilization, the construct was transfected transiently into S2 cells. The transfected cells were then treated with drugs that influence calcium release. It was found that treatment with Thapsigargin (500 nM) or Staurosporine (500 nM) activated the reporter gene by a 5 - 10 fold increase (FIG. 9). Considering previous experience with mammalian reporter systems, these results indicate that the pcFUSII construct can be used as a reporter vector in insect cells.

Test of the Aequorin Based Reporter System in Insect Cell Lines

In order to test the usefulness of this reporter system in insect cells, the pIE1-aequorin expression plasmid was co-transfected with expression vectors for the rat α1b and the CCR5 receptors into Sf9 cells. It was found that the rat α1b receptor was able to transduce calcium mobilization in Sf9 cells using the endogenous G-proteins of the insect cells. CCR5, on the other hand, was able to mobilize calcium only if it was co-transfected with an expression vector that expresses the gene for the human G_{α}16 subunit. Thus, because the present invention provides recombinant cells comprising not only a chimeric reporter construct linked to a human cell surface receptor, but a heterologous human signal transduction pathway as well, the system can be used in a variety of cells using a variety of cell surface receptors.

### Example 10

### Construction of a Promoter Containing a Mixture of different Types of Control Elements.

Construction of the plasmid pcFUS2-6xSTAT/NFKκB was achieved by ligation of the oligonucleotides O9 (5′TTTCCGGGAAATTCCCTTTCCGGGAAATTCCCTTTC CGGGAAATTCCCGGATCC 3′; SEQ ID NO:16) and O10 (5′GGGAATTTCCCGGAAAG GGAATTTCCCGGAAAGGGAATTTCCCGGAAA 3′; SEQ ID NO:17), in two copies each, into the EcoRV digested pcFUS2 vector. The KpnI/XhoI fragment containing the 6xSTAT/NFκB-cassette was excised and ligated to the XhoI restricted pGL3-basic plasmid (PROMEGA) to get pGL3-12xSTAT/NFκB. The reporter plasmid pcFUS3 was constructed by replacing the KpnI/HindIII promoter fragment of pcFUS2-6xSTAT/NFκB with the KpnI/HindIII promoter fragment of pGL3-9xAP-1 FOS instead. The sequence of the promoter of pcFUS3, containing the XhoI/HindIII fragment containing the 6xSTAT/NFκB and the 9xAP-1 cassette is disclosed herein as SEQ ID NO:18.

### Example 11

### Characterization of Mammalian Reporter Cell Lines Transfected with pcFUS3

The reporter vector pcFUS3 was stably electroporated into HeLa cells by standard techniques known in the art. Three hundred twenty stable cell clones were screened by the Ocular Selection Procedure and twenty clones reconfirmed twice with the luciferase assay procedure after PMA or ATP stimulation (as described in Examples 4 and 5). Five of the clones performed superior to HF1 cells in all tests performed. The cell clone most suitable for the purpose was named HFF11 and was used as an exemplary clone for further study.

Endogenously expressed receptors in the HFF11 cells were stimulated with the respective ligands known to the art. The results obtained by stimulating the endogenously expressed receptor CXCR4, with SDF-1 or endogenously expressed ATP-receptors with ATP showed an increased signal-to-noise ratio in respect to HF1 cells by a factor of two to three.

HFF11 cells were used to establish cell lines stably expressing the human CCR5 or the human receptor for C5a (C5aR). After maximal agonist stimulation luciferase activity increased about 80 times in cells transfected with CCR5 and about 30 times in cells transfected with the C5aR stimulated with a C5a C-terminal peptide (BACHEM H-3462).

### Example 12

### Characterization of Mammalian Cell Lines Transiently Transfected with Reporter Constructs

The prototypic reporter plasmids (pcFUS2; pcFUS2-6xSTAT/NFκB; pcFUS3 and pGL3-12xSTAT/NFκB) were used to transfect HeLa cells transiently by electroporation. Cells were stimulated 24 h post transfection with 100 nM PMA or 100 nM PMA and 10⁻⁶ M thapsigargin or treated as controls. After 10 h of incubation, the cells were lysed and assayed. The Amplification values are shown in Table 3.

TABLE 3

| Plasmid name (elements) | Stimulation with | Amplification value |
|---|---|---|
| pcFUS2 (9 × AP-1) | PMA | 2.53 ± 2.00 |
| pcFUS2 (9 × AP-1) | PMA and thapsigargin | 3.30 ± 1.83 |
| pcFUS2-6 × STAT/NFκB | PMA | 3.78 ± 1.44 |
| pcFUS2-6 × STAT/NFκB | PMA and thapsigargin | 5.34 ± 0.78 |
| pcFUS3 (6 × STAT/NFκB; | PMA | 7.37 ± 3.29 |
| 9 × AP-1) | | |
| pcFUS3 (6 × STAT/NFκB; | PMA and thapsigargin | 8.85 ± 4.21 |
| 9 × AP-1) | | |
| pGL3-12 × STAT/NFκB | PMA | 4.16 ± 2.00 |
| pGL3-12 × STAT/NFκB | PMA and thapsigargin | 9.46 ± 4.42 |

The invention has been described in detail above with reference to preferred embodiments. However, it will be understood by the ordinary artisan that various modifications and variations can be made in the practice of the present invention without departing from the scope or spirit of the invention. All references cited herein are hereby incorporated by reference in their entirety.

What is claimed is:

1. A reporter construct comprising a chimeric reporter gene operably linked to at least one transcription control element, wherein said chimeric reporter gene comprises coding sequences from two different genes fused in frame such that each of said coding sequences produces a gene product that is detectable without the need to lyse or otherwise destroy or diminish the viability of the cell in which they are expressed.

2. The reporter construct of claim 1, wherein the chimeric reporter gene comprises coding sequences from a gene encoding a fluorescent protein and coding sequences from a gene encoding a protein that luminesces.

3. The reporter construct of claim 1, wherein the chimeric reporter gene comprises coding sequences from a luciferase gene, an antibiotic resistance gene, a heavy metal resistance gene, or coding sequences from two of these genes.

4. The reporter construct of claim 1, wherein the chimeric reporter gene comprises coding sequences from the firefly luciferase gene, the bacterial luciferase gene, the Renilla luciferase gene, the Photinus luciferase gene, the green fluorescent protein (GFP) gene, the enhanced green fluorescent protein (EGFP) gene, the chloramphenicol acetyl transferase (CAT) gene, the alkaline phosphatase gene, the β-galactosidase gene, or coding sequences from two of these genes.

5. The reporter construct of claim 1, wherein the construct is a plasmid, a virus, a viral nucleic acid, a cosmid, a phagemid, or an artificial chromosome.

6. The reporter construct of claim 1, wherein the chimeric reporter gene comprises sequences from the gene encoding the enhanced green fluorescent protein (EGFP) and the gene encoding the Photinus luciferase.

7. The reporter construct of claim 1, wherein said at least one transcription control element comprises a second messenger-responsive element.

8. The reporter construct of claim 1, wherein said at least one transcription control element is a cAMP responsive element (CRE), a TPA responsive element (TRE; AP-1), an NFAT responsive element, or a mixture of these three elements.

9. The reporter construct of claim 1, wherein said at least one transcription control element is responsive to intracellular signals that can be generated, either directly or ultimately, as a result of binding of a cell surface receptor to a ligand.

10. The reporter construct of claim 9, wherein said at least one transcription control element is responsive to cyclic adenosine monophosphate (cAMP) or phorbol-12-myristate- 13-acetate (TPA).

11. The reporter construct of claim 1, wherein said at least one transcriptional control element comprises multiple TRE motifs fused to a minimal promoter.

12. A recombinant cell comprising
a) a reporter construct comprising a chimeric reporter gene operably linked to at least one transcription control element, wherein said chimeric reporter gene comprises coding sequences from two different genes fused in frame such that each of said coding sequences produces a gene product that is detectable without the need to lyse or otherwise destroy or diminish the viability of the cell in which they are expressed, and
b) a cell surface receptor or ion channel,
wherein interaction of the cell surface receptor or ion channel with a substance that specifically interacts with the receptor or channel modifies the level of expression of the reporter gene.

13. The recombinant cell of claim 12, wherein the cell is a mammalian cell or an insect cell.

14. The recombinant cell of claim 12, wherein the cell comprises a cell surface receptor that is a heptahelix receptor.

15. The recombinant cell of claim 12, wherein the cell is present as a component of a kit.

16. A process for detecting a substance that specifically interacts with a cell-surface receptor protein or ion channel, said process comprising:
a) providing a recombinant cell comprising
i) a reporter construct comprising a chimeric reporter gene operably linked to at least one transcription control element, wherein said chimeric reporter gene comprises coding sequences from two different genes fused in frame such that each of said coding sequences produces a gene product that is detectable without the need to lyse or otherwise destroy or diminish the viability of the cell in which they are expressed, and
ii) a cell surface receptor or ion channel, wherein said cell surface receptor or ion channel is expressed on the surface of the recombinant cell
wherein specific interaction of the cell surface receptor or ion channel with a substance modifies the level of expression of the reporter gene,
b) exposing the recombinant cell to a sample containing at least one substance suspected of being capable of specifically interacting with said cell surface receptor or ion channel, and
c) determining whether expression of the chimeric reporter gene is altered,
wherein alteration of reporter gene expression indicates interaction of a substance in the sample with the cell surface receptor or ion channel, and thus the presence of a substance in the sample that specifically interacts with said cell-surface receptor or ion channel.

17. The process of claim 16, wherein the cell surface receptor is a heptahelix receptor.

18. The process of claim 16, wherein alteration of gene expression is an increase in gene expression.

19. The process of claim 16, wherein said chimeric reporter gene comprises sequences from the gene encoding the enhanced green fluorescent protein (EGFP) and the gene encoding the Photinus luciferase.

20. The process of claim 19, wherein providing a recombinant cell comprises clonal selection by detection of a signal due to the EGFP.

21. The process of claim 20, wherein Fluorescence Activated Cell Sorting (FACS) or fluorescence microscopy is used to detect the signal.
